(19) United States
(12) Patent Application Publication
(10) Pub. No.: US 2023/0383484 A1
(43) Pub. Date: Nov. 30, 2023

(54) METHODS AND TOOLS FOR SUPPORTING TIMING OF CONCRETE PROCESSING OPERATIONS

(71) Applicant: HUSQVARNA AB, Huskvarna (SE)

(72) Inventors: Tobias Gustafsson, Göteborg (SE); Johan Sandwall, Gråbo (SE); Marcus Trybom, Ekerö (SE); Philippe Livernette, Neuville les Dieppe (FR)

(21) Appl. No.: 18/028,319

(22) PCT Filed: Sep. 13, 2021
(86) PCT No.: PCT/SE2021/050873

§ 371 Date: Mar. 24, 2023

(30) Foreign Application Priority Data
Sep. 24, 2020 (SE) 2051109-3

(51) Int. Cl. E01C 23/01; E04F 21/24; G01N 33/38

(52) U.S. Cl. CPC E01C 23/01; E04F 21/245; G01N 33/383; E01C 23/025

(57) ABSTRACT

A concrete processing system (200) for determining an onset of a first time window (301) for a concrete processing operation in a first area (A), the system (200) comprising one or more concrete sensors (220), a data processing system (240) and a timing support tool (250), wherein the one or more concrete sensors (220) are configured to determine a temperature and/or a moisture level of a maturing concrete slab in the first area (A), and to transmit sensor data indicating the temperature and/or moisture level via respective first communication links (230) to the data processing system (240), the data processing system (240) comprising a database (243) and processing circuitry (242) configured to determine a concrete maturity level for the first area (A) based on the sensor data, to estimate the onset of the first time window based on the concrete maturity level and on data associated with the concrete processing operation stored in the database (243), and to transmit timing data indicating the onset of the first time window to the timing support tool via a second communication link (260), wherein the timing support tool (250) is arranged to support timing of the concrete processing operation in the first area (A) based on the timing data.

### TECHNICAL FIELD

The present disclosure relates to concrete processing such as early entry concrete sawing and power troweling. There are disclosed methods, systems and tools for supporting decisions of when to commence a concrete processing operation in response to reaching a sufficient maturity level of a concrete slab after pouring, and optionally also when to cease the concrete processing operation due to that the concrete slab has matured beyond the ideal processing time window.

### BACKGROUND

Early entry concrete sawing is a concrete processing technique where shallow cuts are made in concrete surfaces within the first one or two hours after finishing surface preparation, i.e., after the concrete has reached a certain level of maturity but before the concrete has set completely, a period often referred to as the ‘green zone’. The shallow cuts create a weakened surface plane, thus encouraging cracking to occur at the location of the cuts in a controlled manner rather than the cracks appearing anywhere in the concrete, resulting in a more visually appealing surface. One example of early entry concrete saws are the Soff-Cut range of early entry concrete saw products manufactured by Husqvarna AB.

Power troweling is another concrete processing technique where a power trowel or power float is used to apply a level surface and a smooth finish to a concrete slab. Just as for the early entry sawing, there is a first time window when the concrete has set enough to be processed efficiently by the trowel, but not too much as to make the troweling difficult. Power troweling is often performed in steps, i.e., panning, followed by floating and then finishing. Each power troweling step is associated with an ideal time window where the concrete has matured just enough to support the operation in an efficient manner but not too much so as to make the concrete hard and difficult to process.

Concrete processing contractors today depend largely on professional knowledge and experience gathered by years of working with concrete. There is a saying in the industry that goes “concrete does not wait for you, you wait for the concrete”.

US 2017370898 A1 discloses a system for assisting a contractor in performing a concrete processing operation. The system permits a user to configure a concrete maturity level in terms of an “action PSI”, where a concrete processing operation may suitably commence.

However, despite the advancements to-date, there is a need for further improvements in tools and methods which allow an even more efficient concrete processing.

### SUMMARY

It is an object of the present disclosure to provide a concrete processing system for determining an onset of a first time window for a concrete processing operation in a first area. The system comprises one or more concrete sensors, a data processing system, and a timing support tool. The one or more concrete sensors are configured to determine a temperature and/or a moisture level of a maturing concrete slab in the first area, and to transmit sensor data indicating the temperature and/or moisture level via respective first communication links to the data processing system. The data processing system comprises a database and processing circuitry configured to determine a concrete maturity level for the first area based on the sensor data, to estimate the onset of the first time window based on the concrete maturity level and on data associated with the concrete processing operation stored in the database, and to transmit timing data indicating the onset of the first time window to the timing support tool via a second communication link, whereby the timing support tool is arranged to support timing of the concrete processing operation in the first area based on the timing data.

This way a machine operator, a contractor, or a project planning engineer can determine when a given processing operation can commence, due to that the concrete surface segment has reached a sufficient maturity level. This is an advantage since there is a reduced need to rely on experience to deem when a given concrete processing operation can start. It is a particular advantage that the estimation of time window onset is made in dependence of data associated with the particular processing operation, since this means that the particulars of the operation can be accounted for. The system not only predicts in real-time when an operation can commence, but also allows for the prediction of a future onset of the time window for the concrete processing operation. For instance, the data processing system can be configured to generate extrapolated temperature and/or moisture level data from the sensor data, and to predict a future onset and/or a future cessation of the first time window. Thus, planning of a sequence of different concrete processing operations is facilitated since there is no longer a requirement to wait for the concrete until the “feeling” is right. Instead, an operator can receive information indicating that, e.g., given the circumstances and the planned processing operation, it is estimated that processing can start tomorrow at noon.

The database may, e.g., be built by gathering information from concrete processing operations performed by experienced operators. The temperature and/or moisture level of the maturing concrete slab is then recorded when the experienced operator begins processing the concrete with a particular tool. Given multiple such data points gathered over time, a statistic can be computed such as an average, a minimum, a maximum, or some other statistical measure of temperature and/or moisture level when a given processing operation is started. The system then measures the current temperature and/or moisture level of a maturing concrete slab, and when the measured values has reached a level corresponding to the data points in the database, onset of the first time window is declared. Advantageously, there is no need for the experienced operators to input data about temperatures or moisture levels required to start processing.

The database can also be populated with data points indicating when the experienced operators stop processing the concrete slab with a given tool. These data points can be used in a similar manner to determine when the first time window ends.

The database can furthermore be populated with data from computer simulation of maturing concrete slabs, as will be discussed in more detail below.

According to aspects, the data associated with the concrete processing operation comprises data associated with a particular type of concrete processing tool or machine and/or a particular type of concrete processing operation. This brings the additional advantage of customizing the time window onset predictions to the particulars of the concrete processing operation. For instance, some machines may be able to process less mature concrete surface segments than other machines due to larger supporting wheels, or the lack of an operator leaving foot-prints in the concrete surface in case the machine is configured for autonomous drive, even if the actual processing operation is the same.

According to aspects, the data associated with the concrete processing operation stored in the database comprises information related to previous concrete processing operations. These are the data points discussed above, i.e., the measured temperatures and/or moisture levels when previous concrete processing operations where started. This way the database may assume the role of the experienced operator “gut feeling” for when to start processing. The previous concrete processing operations can be processing operations using a particular type of concrete processing tool or machine and/or a particular type of concrete processing operation. This way the database also reflects different tool characteristics that the experienced operators are intuitively aware of, but which may be difficult to explain to the inexperienced operator. For instance, differences in tool weight may have an impact on the first time window. By recording the actions of several experienced operators, the database enables estimation of suitable time windows for when to perform a given concrete processing operation. The estimated time window essentially mimics the decisions taken in the past by the experienced operators.

According to aspects, the information related to the previous concrete processing operations comprises one or more statistics of onset of the processing operation with corresponding concrete temperature and/or a moisture level.

According to aspects, the timing support tool comprises display means arranged to indicate onset of the first time window to a user of a concrete processing tool in the first area. Thus, in its perhaps simplest form, a concrete processing machine can be equipped with a green and a red light, and the operator just has to wait for the green light to go on before processing can start. This way less experienced operators can be employed, and the risk of performing a concrete processing operation at the wrong time due to human error is reduced. The display means can also be more advanced, indicating also for instance the time to go before onset of the first time window, such that the operator can plan operations more efficiently instead of just waiting on site for the concrete to become sufficiently hard. Although perhaps simple in realization, the green light essentially represents perhaps tens of years or experience or more, by drawing on the information stored in the database.

According to aspects, the processing circuitry is arranged to trigger the concrete processing operation in the first area at the onset of the first time window by the timing support tool. The concrete processing systems disclosed herein can be advantageously used together with autonomous concrete processing machines, such as autonomous Soff-cutting equipment and power trowels. In such systems, the processing circuitry may transmit a signal to the autonomous processing system indicating that it is time to start processing.

According to aspects, the data processing system is also arranged to determine a cessation of the first time window based on the concrete maturity level and on data associated with the concrete processing operation stored in the database, and to transmit timing data indicating the cessation of the first time window to the timing support tool via a second communication link. The data regarding cessation of the time window can be used to further refine planning of a project. Some processing operations may be associated with longer duration time windows compared to other processing operations. Thus, some operations may be put on hold for some time without jeopardizing the overall project. The data regarding cessation of the first time window can also be used to prevent concrete processing operations outside of the time window. Thus, tool wear from processing too hard concrete can be avoided, thereby preventing shortened tool lifetime. For instance, the processing circuitry can be arranged to generate a warning signal, or even to prevent a concrete processing operation in the first area by the timing support tool before the onset of the first time window and/or after cessation of the first time window.

According to aspects, the data processing system is configured to determine the concrete maturity level by indexing a maturity level table stored in the database using the sensor data. This maturity level table can be built up over time, thus gathering valuable experience.

According to aspects, the data processing system is configured to determine the onset of the first time window in dependence of a pre-determined maturity level requirement of the concrete processing operation. This means that the onset of the time window can be influenced by machine equipment providers and vendors. For instance, the onset of the time window can be configured in order to maximize tool life-time, which may be desired by a tool rental agency. The onset of the time window can also be configured to maximize production rate and throughput, which may be desired by a contractor or project planner.

According to aspects, the data processing system is configured to obtain a recipe of the concrete used in the concrete slab in the first area, and to determine the concrete maturity level in dependence of the recipe. By basing the determination of concrete maturity level also on concrete recipe, the maturity estimation can become more accurate, since various additives and the chemical composition of the concrete can be accounted for in a more accurate manner.

According to aspects, the data processing system is configured to obtain weather forecast data and/or data from one or more weather stations deployed in connection to the first area, and to predict a future concrete maturity level in dependence of the weather forecast data and/or in dependence of the data from the one or more weather stations. This way the determined concrete maturity level and the predictions of future concrete maturity levels become more accurate, since different current and/or future weather conditions can be accounted for by the system.

The database may, according to an example, be configured to be populated by data from empirical testing of concrete processing operations. This way it becomes possible to base future processing operations on the outcome of past processing operation, thereby gathering experience. In other words, the system can learn from past experiences in order to optimize future processing operations.

The concrete processing systems disclosed herein can optionally also be configured for determining an onset and/or a cessation of a second time window for a concrete processing operation in a second area, wherein the one or more concrete sensors are configured to determine a temperature and/or a moisture level of a maturing concrete slab in the second area, and to transmit sensor data indicating the temperature and/or moisture level via respective first communication links to the data processing system, wherein the data processing system is configured to determine a concrete maturity level for the second area based on the sensor data, to estimate the onset of the second time window based on the concrete maturity level and on data associated with the concrete processing operation stored in the database, and to transmit timing data indicating the onset of the second time window to the timing support tool via a second communication link, wherein the timing support tool is arranged to support timing of the concrete processing operation in the second area based on the timing data. Thus, it is understood that the support systems disclosed herein can be used for planning and performing different types of concrete processing operations at one or more sites, comprising one or more areas. The system determines time windows for each processing operation and for each area, such that an operator can know when and where it is and/or will be possible to process the concrete with a given type of equipment. Based on this information it is possible to improve work schedules significantly, since much smaller time margins are necessary.

The concrete processing systems discussed herein can be configured to determine at least respective onsets of time windows for any of panning, floating, and finishing power troweling concrete processing operations, and an early entry sawing concrete processing operation.

According to aspects, the concrete processing system comprises a plurality of concrete sensors arranged distanced from each other over the first area. The processing circuitry can then be configured to determine the concrete maturity level for the first area based on the concrete sensor reporting data indicating the lowest maturity level. This way variations in the maturing rate of a concrete surface segment can be accounted for. It is understood that some areas may mature faster than others, due to, e.g., that the sun shines on some areas and not on some other areas. However, by considering the sensor reporting the lowest degree of maturity more than the others, such effects can be compensated for. This way too early processing can be avoided, even if some sensors report data indicating high degrees of concrete maturity. Optionally, the processing circuitry can also be configured to estimate the onset of the first time window based on a variation in the sensor data from the plurality of distanced concrete sensors. Thus, if variation is large, then time margins can be increased to account for the variation.

The concrete sensors are preferably deployed at a depth between 2-15 cm from the concrete surface level, and more preferably between 2-5 cm measured from the concrete surface level. This is because the concrete maturity level at the surface is important for concrete processing operations such as early entry sawing and power troweling.

According to aspects, the data processing system is arranged to generate log data pertaining to the first time window and to the concrete processing operation. This log data can be used for, e.g., investigating cause of damages and the like, and also to generate proof that a concrete processing operation has been performed according to specification, and not outside of the time window. For instance, the data processing system can be arranged to generate a receipt and/or a certificate in case the concrete processing operation is performed within the first time window. The data processing system is optionally also arranged to generate a warning and/or an alarm signal in case the concrete processing operation is performed outside of the first time window. This way a tool rental agency of contractor can be made aware is a sub-contractor or operator has performed a concrete processing operation outside of the time window and take appropriate action in response to the warning or alarm signal.

There are also disclosed herein data processing systems, timing support tools, concrete processing equipment such as early entry concrete saws and power trowels, as well as methods associated with the advantages mentioned above.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated. Further features of, and advantages with, the present invention will become apparent when studying the appended claims and the following description. The skilled person realizes that different features of the present invention may be combined to create embodiments other than those described in the following, without departing from the scope of the present invention.

### BRIEF DESCRIPTION OF THE DRAWINGS

The present disclosure will now be described in more detail with reference to the appended drawings, where

FIGS. 1A, B show example concrete processing equipment;

FIG. 2 schematically shows a concrete processing support system;

FIG. 3 is a timing diagram illustrating message exchange in a concrete processing system;

FIG. 4 is a graph illustrating a concrete maturing process;

FIGS. 5,6 schematically illustrate example timing support tool interfaces;

FIG. 7 is a flow chart illustrating methods;

FIG. 8 schematically illustrates a control unit; and

FIG. 9 schematically illustrates a computer program product.

### DETAILED DESCRIPTION

The invention will now be described more fully hereinafter with reference to the accompanying drawings, in which certain aspects of the invention are shown.

This invention may, however, be embodied in many different forms and should not be construed as limited to the embodiments and aspects set forth herein; rather, these embodiments are provided by way of example so that this disclosure will be thorough and complete, and will fully convey the scope of the invention to those skilled in the art. Like numbers refer to like elements throughout the description.

It is to be understood that the present invention is not limited to the embodiments described herein and illustrated in the drawings; rather, the skilled person will recognize that many changes and modifications may be made within the scope of the appended claims.

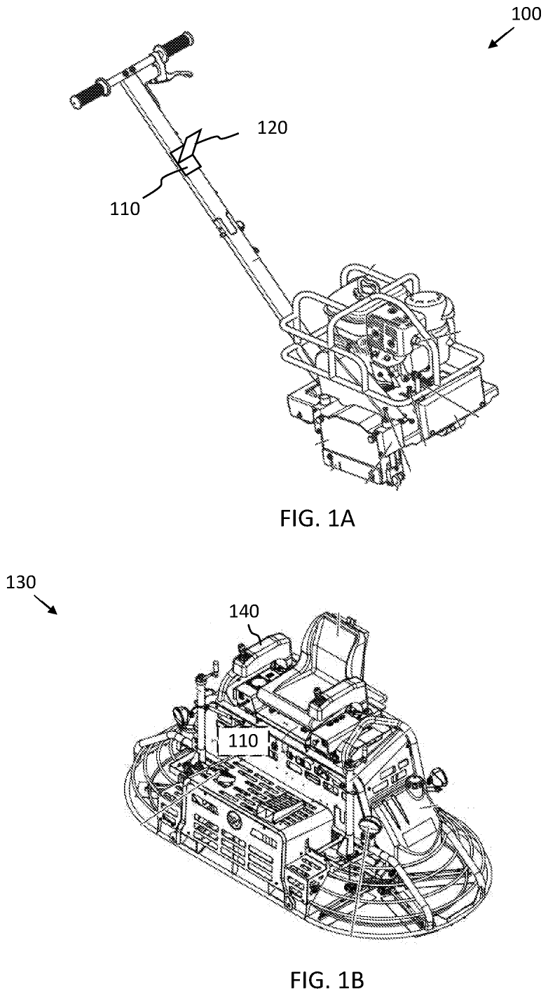

FIG. 1A illustrates an early entry concrete saw 100. As mentioned above, this type of saw is used to cut shallow cuts in concrete surfaces after finishing surface preparation, but before the concrete has set completely.

A concrete slab has a concrete surface. The part of the concrete slab immediately under the concrete surface is herein referred to as the concrete surface segment. The thickness of this segment varies, but may be on the order of 10-15 cm or so.

FIG. 1B illustrates an example power trowel. Just like the early entry concrete saw, there is an ideal time window for each power troweling step, where the concrete has matured just enough to support the concrete processing operation but not too much so as to become too hard. If the concrete has not matured enough when commencing a troweling operation, the result will not be ideal since the concrete will be too soft. Trying to power trowel too mature concrete is time consuming and causes excessive tool wear. Thus, there is a time window where power troweling is ideally performed.

Today users depend on professional knowledge gathered by years of working with concrete and in some cases supporting estimates from documentation by, e.g., the American Concrete Institute (ACI) to know when to enter concrete with a Soft-Cut saw or a power trowel.

The present disclosure relates to a concrete processing system which comprises a database where data associated with various past concrete processing operations has been stored. Conceptually, the database may for instance be populated with empirical information about at which estimated maturity level that experienced contractors and machine operators determined that the time was right to start processing the concrete with a given machine, such as an early entry concrete saw or a power trowel, in the past. This allows the system to inform inexperienced operators about when the time is right to start processing the concrete with a given tool, based on the data associated with the given concrete processing operation stored in the database, simply because the current concrete maturity has reached the level where the experienced operators started processing the concrete in the past.

The invention may, e.g., be realized as a green light attached to the handle of an early entry saw. The inexperienced operator then only has to wait for the green light to turn on, and then commence the concrete processing operation. In essence, that green light assumes the role of the “gut feeling” of an experienced operator.

It is appreciated that there are differences between different types of machinery when it comes to the ideal time window or maturity range for optimal processing, and even between equipment for the same task. Thus, two different models of power trowel may be associated with different ideal time windows for concrete processing operations. Known systems for estimating concrete maturity based on sensor data does not account for the particular concrete processing operation to be performed, nor do they learn over time to become more and more accurate at estimating the ideal time slot for a given concrete processing operation.

The present disclosure relates to a system for estimating suitable time windows for various concrete processing operations which relies on a combination of real time sensor data and data from previous concrete processing operations and trials stored in a database.

The system operates based on sensor data gathered by one or more sensors arranged in connection to or embedded within the maturing concrete slab. The sensor data obtained from these sensors are fed into a data processing system which estimates a concrete maturity level based, e.g., on temperature, moisture level, concrete recipe and so on. The data processing system also has access to the database which comprises concrete processing data obtained from practical experience with different types of machines, different operations, and/or different types of concrete, as well as from computer simulation and laboratory experimentation. The database can be used to translate between an estimated maturity level of the concrete and a time window where a given concrete processing operation (potentially by a specific type of machine) can be performed, e.g., an ideal cutting timeframe for early entry sawing or time windows for when the three steps of power troweling are ideally performed by a certain type of power trowel. By feeding in empirical test data to the database, the database can with time assume a role similar to that of the experienced contractor who intuitively knows when it is time to start a given concrete processing operation in a given area when using a certain type of equipment.

According to an example, the database can be populated with datapoints indicating at which temperature and/or maturity level that a given concrete processing operation was started by an experienced contractor. The datapoints may be obtained by the same type of sensor as used in the disclosed system, or by some other form of sensor. Each datapoint can then be associated with a given tool and/or a given construction scenario. Once the database comprises a sufficient number of such datapoints, a statistical measure can be computed, such as an average temperature and/or moisture level when a given concrete processing operation was started. This statistic can then be used to determine the start of a time window for performing the concrete processing operation.

The same idea can be applied to determining the end of the time window for when concrete processing can be performed. The database can be populated with datapoints indicating at which temperature and/or maturity level that a given concrete processing operation was stopped by an experienced contractor. The database then comprises data for when the concrete processing operation is no longer possible to perform, since the concrete has reached a too high level of maturity.

In this case, the “experienced contractor” may, e.g., be a contractor handpicked for his or her known skills, or a contractor which has some form of credentials or certification.

The data processing system is arranged to interface with one or more timing support tools that assist the concrete workers by indicating when it is time to start a given concrete processing operation in some area. These timing support tools may be configured to indicate in which sub-area in a larger area a concrete processing operation may commence, and thus present a valuable tool when it comes to planning a sequence of concrete processing operations over a larger area.

The timing support tool can also interface with systems for autonomous or semi-autonomous operation of concrete processing tools such as autonomous early entry concrete saws and automated power trowels. More advanced systems may trigger autonomous concrete processing in some area when the time is just right for the concrete processing operation at hand to commence, while other, less advanced systems may simply indicate by a green light or the like arranged on the machine when it is time to start processing, thus guiding the operator by visual cue or via some sort of display means. The systems may also be configured to dissuade an operator from processing concrete outside of the ideal time window, or even prevent concrete processing outside of the estimated time window by locking down or stopping the concrete processing equipment if the time is not right for that particular type of processing.

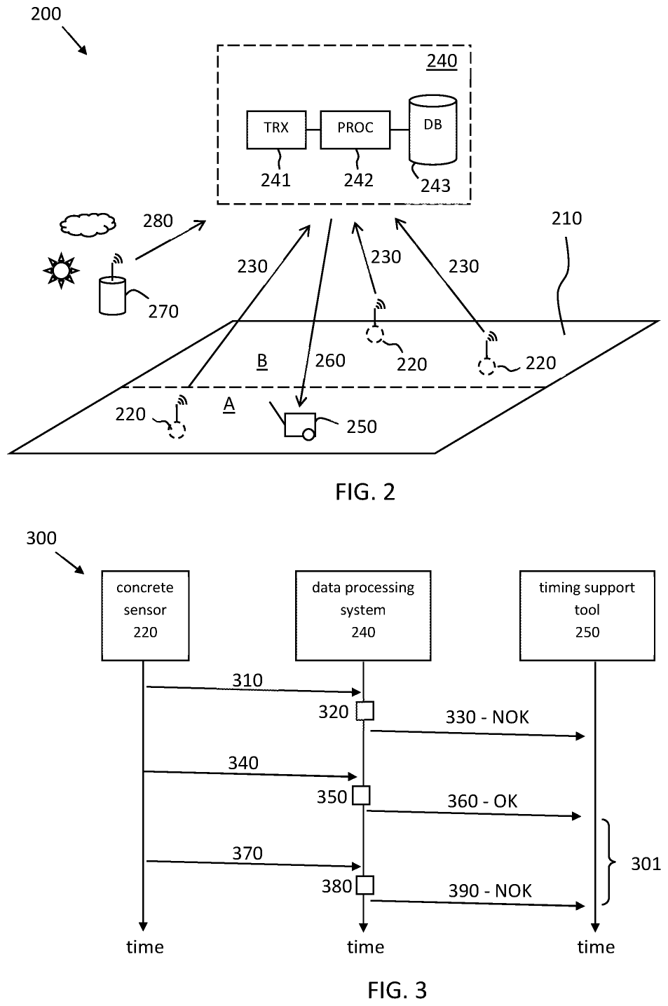

FIG. 2 schematically illustrates the proposed concrete processing system 200 for determining an onset of a first time window for a concrete processing operation in a first area A. The system 200 comprises one or more concrete sensors 220, a data processing system 240 and a timing support tool 250, which timing support tool may be integrated with a concrete processing machine or realized as a handheld terminal for communicating information to an operator.

The one or more concrete sensors 220 are configured to determine a temperature and/or a moisture level of a maturing concrete slab in the first area A, and to transmit sensor data indicating the temperature and/or moisture level via respective first communication links 230 to the data processing system 240. Concrete sensors 220 for determining the temperature and/or the moisture level in concrete are known and will therefore not be discussed in more detail herein. However, known concrete sensors fail to account for variations in concrete maturing processes in relation to characteristics of specific types of tools. One type of power trowel may, for instance, require a less matured concrete slab for commencing operations while another power trowel which performs essentially the same function perhaps requires a higher degree of concrete maturity before it is ideal to start the power troweling process.

The sensor data indicating the temperature and/or moisture level may comprise a direct representation of temperature in, e.g., Fahrenheit, or Celsius and/or moisture level measured in, e.g., moisture content percentage. However, the sensor data may also just be some proprietary number on a scale ranging from low temperature to high temperature, and/or high moisture content to low moisture content.

According to some aspects, the sensor data forms the waist portion of an auto-encoder artificial intelligence network configured to determine suitable time windows for a number of different concrete processing operations based on the sensor data. An auto-encoder is a type of artificial neural network used to learn efficient data encodings in an unsupervised manner. The aim of an auto-encoder is to learn a representation (encoding) for a set of data, typically for dimensionality reduction, by training the network to ignore signal “noise”. Thus, by representing sensor data by an auto-encoder network, the sensor data becomes efficiently encoded for transport over the first communication links 230.

The data processing system 240 comprises a transceiver 241 for communicating with the sensor devices 220 over a wireless (or wireline link). This wireless link may, e.g., form part of the Internet or be a proprietary communications protocol.

The data processing system 240 also comprises a database 243 as discussed above and processing circuitry 242 configured to determine a concrete maturity level for the first area A based on the sensor data. The database 243 is key to many of the advantages obtained from the disclosed system 200, since it allows for storing results from, e.g., field trials through which ‘experience’ can be collected.

The concrete maturity level can be estimated from the sensor data since the temperature and the moisture content in the concrete slab changes over time in a maturing concrete slab. Thus, according to some aspects, the data processing system 240 is configured to determine the concrete maturity level by indexing a maturity level table stored in the database 243 using the sensor data. This maturity level table can be populated by both analytical data and experimental data over time in order to provide a robust and accurate estimate of concrete maturity level.

The data processing system 240 is optionally also configured to obtain a recipe of the concrete used in the concrete slab in the first area A, and to determine the concrete maturity level in dependence of the recipe. This way variation in maturing rates which are due to variations in the concrete recipe, and effects from different additives in the concrete, can be accounted for in order to further improve the accuracy of the concrete maturity determination. The recipe data can be pre-configured or obtained on demand by, e.g., user input.

The data processing system 240 is arranged to estimate the onset of the first time window based on the concrete maturity level and on data associated with the concrete processing operation stored in the database 243. As discussed above, the database comprises data indicative of when similar concrete processing operations were started in the past, optionally also using similar or identical concrete processing tools, by experienced operators performing similar concrete processing tasks under similar circumstances. For instance, if the concrete processing operation to be performed is early entry sawing using a certain equipment E1, then the database 243 may indicate a certain time instant as suitable for commencing with the concrete processing operation based, e.g., at least in part on previous field trials involving the equipment E1. However, if the user instead has some other piece of concrete sawing equipment, say equipment E2, then the “experience database” may instead indicate a slightly later onset as ideal due to that this particular saw is better suited for cutting into slightly more mature concrete. This becomes possible since the database draws on the experience from past concrete processing operations. In this way, practical experience gathered over time with different types of tools can be stored in the database and used to refine the estimated onset of the ideal processing time duration.

According to another example, the concrete processing operation is surface sawing by a regular floor saw. This operation has the same purpose as early-entry sawing, i.e., to control crack formation in the concrete surface, but floor sawing can be performed later on more mature concrete compared to a Soff-cut operation which requires operation in the green zone mentioned above. Depending on the floor saw equipment, such as its weight, supporting wheels and whether the machine is self-propelled or manually pushed, varying levels of concrete maturity are required in order to support the floor saw and the floor saw operator without leaving marks in the concrete surface. For instance, an operator may leave marks even in relatively mature concrete slabs, while an autonomous floor saw without operator can normally enter a concrete slab sooner, i.e., while it is still relatively soft. The concrete processing systems disclosed herein also support timing of floor sawing operations. By estimating the onset of the first time window based on the concrete maturity level and on data associated with the floor sawing operation such as machine type and whether an operator will also enter the concrete slab surface, planning of concrete processing operations is facilitated.

When using a power trowel, e.g., to float or finish a concrete surface, the operator normally walks in front of the machine to smooth out any footprints. Thus, the onset of the first time window will not be so much affected by whether the power trowel operation involves an operator or if the machine is autonomous.

The database 243 may be updated continuously by a feedback mechanism which allows users to feed back results of concrete processing operations performed according to the time windows proposed by the system 200. For instance, if some operation started too soon despite the system 200 reporting an open time window for that given concrete processing operation, then this can be reported back to the data processing system 240 which may adjust the database 243 to delay future proposed onsets somewhat in response to the feedback. Also, if a contractor thinks that the proposed time window started too late, i.e., that the concrete was too mature at the onset of the time window for a given equipment type and concrete processing operation, then the database 243 can be adjusted to move future proposed onsets to slightly earlier points in time, i.e., shifting the time window forward in time.

For example, suppose that data of temperatures and moisture levels at which a given type of processing operation has been started in the past has been collected in the database. The system may determine a metric m, e.g., as

m=w₁T+w₂M

where T is the average temperature value at which operations were started, and M is the average moisture level at which operations were started, and where w₁ and w₂ are pre-determined weights indicating a perceived relative importance of temperature compared to moisture level in the maturing concrete. The system can then use the metric m to estimate the onset of the first time window based on the measured current concrete maturity level by comparing a test metric m′ to the metric in the data base, where the test metric may, e.g., be determined as

m′=w₁T′+w₂M′

where T′ and M′ are the current output from the concrete sensors (220).

Of course, a wide variety of different metrics can be contemplated, based on averages, maximum values, minimum values, cumulative values, and so on.

The metric may also comprise the output from a machine learning structure, such as a neural network, which has been trained to determine the onset of the first time window based on the concrete maturity level and on data associated with the concrete processing operation stored in the database, by training using the previously collected data of past concrete processing operations performed by experienced operators.

The concrete processing system 200 may be configured to determine at least respective onsets of time windows for any of panning, floating, and finishing power troweling concrete processing operations, and an early entry sawing concrete processing operation. Thus, an operator may receive information indicating different time slots for when various concrete processing operations are suitable to perform, e.g., when the different power troweling operations can be performed with expectation on success, followed by the time window when early entry sawing can be performed.

The data processing system 240 is also arranged to transmit timing data indicating the onset of the first time window 301 to the timing support tool via a second communication link 260. The second communication link 260 may be a wireless link or a combination of a wireless and a wireline link. The data processing system 240 may, e.g., be configured to be access via the internet, in which case both the first and the second communication links 230, 260 comprise access network links. The timing support tool may be configured in connection to display means 120, 140 arranged on the concrete processing equipment, from which an operator can receive an indication of when it is time to start a particular concrete processing operation.

Other types of timing support tools, in particular handheld terminals comprising functionality for assisting in planning a sequence of concrete processing operations over a larger area potentially comprising more than one zone, will be discussed in more detail below in connection to FIG. 5 and FIG. 6.

According to some aspects, the concrete processing system 200 comprises a plurality of concrete sensors 220 arranged distanced from each other and distributed over the first area A. It is appreciated that the concrete slab may mature at different rates at different places in the area A. To ensure that the whole concrete surface segment is ready for a given concrete processing operation, the concrete sensor indicating the lowest grade of maturity can be considered or can be considered more than the other sensors. For instance, a weighted concrete maturity estimate can be formed by weighting the different sensors in relation to their reported data, such that the concrete sensor reporting sensor data indicating the lowest grade of concrete maturity is weighted more than the sensor reporting sensor data associated with the highest grade of concrete maturity. Thus, the processing circuitry may be configured to determine the concrete maturity level for the first area A based on the concrete sensor reporting data indicating the lowest maturity level.

According to some aspects, the concrete processing system 200 comprises a plurality of concrete sensors 220 arranged distanced from each other over the first area A. It is appreciated that the concrete sensors may report data having a variation or variance in the reported sensor data. I.e., some sensors may report data indicating a high degree of concrete maturity while other sensors report data indicating a much lower degree of maturity. This variation in reported sensor data may indicate an uneven degree of concrete maturity over the concrete surface segment in the area A. To account for this variation in maturity, the ideal processing time windows can be shortened by adding a safety margin to account for the level of variation in the concrete sensor data. Thus, the processing circuitry 242 is optionally configured to estimate the onset of the first time window 301 based on a variation in the sensor data from the plurality of distanced concrete sensors. The added safety margin may, e.g., be such that at least 90% of the concrete sensors are reporting sensor data in agreement with the determined time window, and 10% of the concrete sensors are reporting sensor data not in agreement with the determined time window.

According to some aspects, one or more of the concrete sensors 220 are deployed at a depth between 2-15 cm from the concrete surface, and preferably between 2-5 cm measured from the concrete surface. This shallow deployment provides more accurate sensor data regarding maturity conditions at the surface of the concrete slab, where concrete processing operations such as power troweling, and early entry sawing are performed. Maturity data indicating concrete status at higher depth does not affect such concrete surface processing operations as much and is therefore not as relevant in the current context.

An application where the disclosed concrete processing system can be put to use with advantage is a monitoring and logging system for performed concrete processing operations, e.g., at a construction site. The data processing system 240 may be arranged to generate log data pertaining to the first time window and to the corresponding concrete processing operation. For instance, the data processing system 240 may store information related to performed concrete processing operations such as the different types of processing, which machines were used, and so on, along with data pertaining to when each operation was performed, or equivalently at which estimated concrete maturity level. The log data can be stored in the database 243 or elsewhere. A third party can then access the log data to verify if a given concrete processing operation was performed at the right time, or if an error in the timing of a given concrete processing operation has been made. This log data can be used to determine who is at fault in case something goes wrong, for instance of marks are made in the concrete slab, or if cracks form despite early entry sawing according to the specified cutting layout. The data processing system 240 is optionally also arranged to generate a warning and/or an alarm signal in case the concrete processing operation is performed outside of the first time window. This alarm signal can be dispatched to a foreman on-site or some other controlling party. The data processing system 240 may furthermore be arranged to generate a receipt and/or a certificate in case the concrete processing operation is performed within the first time window. This receipt and/or certificate can be presented by a contractor as proof that the concrete processing operation was performed according to specification, and, e.g., not started before the concrete was sufficiently matured.

FIG. 3 is a signaling diagram 300 indicating message exchange between the different entities in the system 200. The concrete sensor 220 regularly or on demand reports sensor data 310, 340, 370 to the data processing system 240. Each time the data processing system 240 receives new sensor data a maturity level of the concrete slab is updated by processing the sensor data 320, 350, 380. Each data processing operation 320, 340, 370 results in a determination of if the concrete processing time window has commenced. In the example shown in FIG. 3, the first report 330 to the timing tool indicates that it is not yet time to start the processing, since the concrete has not yet matured sufficiently for the task at hand (and optionally for the configured concrete processing equipment). The second report 360, however indicates to the timing support tool 250 that the time window is now open, and that the concrete processing operation may commence. Thus, this message indicates an estimated onset of the first time window based on the concrete maturity level and on data associated with the concrete processing operation stored in the database 243. The next message 390 shown in FIG. 3 indicates that the time window 301 has closed. Thus, the concrete processing operation can no longer be efficiently performed without risk of excessive tool wear and a prolonged processing operation.

The system can be used with a single area A, or with more than one area, i.e., with a first area A and a second area B as shown in FIG. 2. The disclosed system may, advantageously, be used in supporting planning of a sequence of concrete processing operations, since the data processing system may be configured to predict suitable time windows for different processing steps based on future predicted concrete maturity levels, as will be discussed in more detail below.

According to some aspects, the data processing system 240 is configured to determine the onset of the first time window 301 in dependence of a pre-determined maturity level requirement of the concrete processing operation.

This pre-determined maturity level requirement can be configured and refined over time for different types of equipment and even for different individuals in a fleet of concrete processing equipment, i.e., some types of equipment may prefer slightly softer concrete due to equipment age, while other individuals can be used during a longer time window since they are able to handle slightly harder concrete. The database 243 can be used to accumulate results from past concrete processing operations, trials, and other experiments such as data from empirical testing of concrete processing operations, as well as the type of feedback from operators and other discussed above. Thus, over time, a sufficiently large database 243 can be built which can be used to optimize timing for different concrete processing operations using different types of concrete processing tools, and even individual machines in a fleet of concrete processing tools of the same type.

According to some aspects, the data processing system 240 is also arranged to determine a cessation of the first time window 301 based on the concrete maturity level and on data associated with the concrete processing operation stored in the database 243, and to transmit timing data indicating the cessation of the first time window 301 to the timing support tool via a second communication link 260.

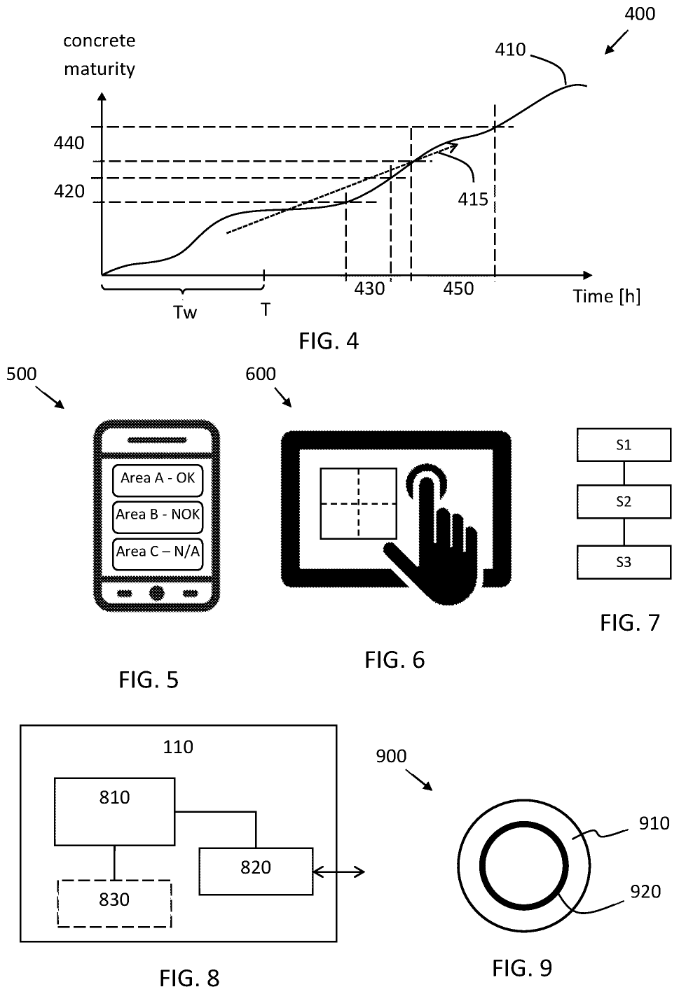

The sensor data reported over the first communication links 230 to the data processing system 240 by the concrete sensors 220 indicates a current maturity state of the concrete slab. However, in some situations is may be advantageous to be able to estimate a future maturity state of a concrete slab. FIG. 4 is a graph 400 which illustrates concrete maturity 410 over time, on a time scale of several hours. Concrete maturity can be measured, e.g., as a percentage relative to total maturity or to some other reference value. The maturing process is seldom an entirely linear process. Instead, the rate of change often varies over time. Nevertheless, its rate of change can be estimated, e.g., by low-pass filtering the sensor data or by applying more advanced signal processing operations such as a Kalman filter configured to estimate the evolution over time of the concrete maturity, e.g., as an average rate of change 615. In other words, at time T, sensor data in a time window Tw has been collected from the concrete sensors. This sensor data sequence can be used to predict future concrete maturity levels, and thereby estimate when a concrete processing operation may commence with expectation on success. It is thus understood that the concrete processing systems 200 discussed herein may be configured to generate extrapolated temperature and/or moisture level data from the sensor data, and to predict a future onset and/or a future cessation of the first time window or also further time windows related to other concrete processing operations. This enables a timing support tool which is able to not only indicate when it is time to start a concrete processing operation, but also how long time remains until the time window opens for processing a given area. Thus, a contractor can receive information of upcoming different time slots for various concrete processing operations in different areas, thus greatly simplifying planning.

A given piece of concrete processing equipment may be associated with a first range 420 of concrete maturity when processing is at its best, this range corresponds to a first time window 430 with an onset and a cessation time instant. It is appreciated that the onset time instant and the cessation time instant are not necessarily determined on a second-basis. Rather the onset and cessation can be given in more approximate terms, e.g., on a half-hour basis or the like.

Another piece of concrete processing equipment, or other concrete processing operation, may be associated with another ideal range 440 of concrete maturity (possibly even overlapping the first range 420), this range then corresponds to a second time window 450.

According to some aspects, the data processing system 240 is configured to obtain weather forecast data, and to predict a future concrete maturity level in dependence of the weather forecast data. Using weather forecast data to improve on the predicted future maturity levels, i.e., on the rate of change in maturity over time, means that the prediction becomes more accurate. For instance, warm weather normally has a positive effect on concrete maturing rate. Of course, the system may also comprise one or more weather stations 270 as schematically illustrated in FIG. 2. This weather station 270 or weather stations may be configured to measure any of: temperature, humidity, amount of rain, and the like, and to report this data 280 to the data processing system 240. The data processing system can then base the estimation of concrete maturity level also on real-time weather data, which is an advantage since different current weather conditions can be accounted for to improve both the current estimate of concrete maturity level as well as predicted future levels of concrete maturity. FIG. 2 also shows a weather station 270 configured to measure, e.g., temperature, humidity, and rainfall. This data 280 can be used to further refine the predictions of future concrete maturity levels, as well as the determination of current concrete maturity level.

With reference to FIGS. 1A, 1B, 2, 5 and 6, the timing support tool 110, 250, 500, 600 is arranged to support timing of the concrete processing operation in the first area A based on the timing data. This can be done in many different ways. For instance, the timing support tool may be arranged to indicate onset of the first time window 301 to a user of a concrete processing tool in the first area A, i.e., by visual indication on a display.

The timing support tool may also comprise a software module for controlling the actual concrete processing equipment. The processing circuitry 242 may then be arranged to remotely trigger the concrete processing operation in the first area A at the onset of the first time window by the timing support tool.

The processing circuitry 242 may also be arranged to generate a warning signal, and/or to prevent concrete processing operation in the first area A by the timing support tool before the onset of the first time window and/or after cessation of the first time window.

With reference to FIG. 2, a larger area may be divided into two or more smaller areas. FIG. 2 shows two such areas A and B. If the data processing system 240 has access to information regarding which sensors that are located in the different areas, and possibly also where in the area, the concrete processing system 200 can be configured for also determining an onset and/or a cessation of a second time window for a concrete processing operation in a second area B, wherein the one or more concrete sensors 220 are configured to determine a temperature and/or a moisture level of a maturing concrete slab in the second area B, and to transmit sensor data indicating the temperature and/or moisture level via respective first communication links 230 to the data processing system 240, wherein the data processing system 240 is configured to determine a concrete maturity level for the second area B based on the sensor data, to estimate the onset of the second time window based on the concrete maturity level and on data associated with the concrete processing operation stored in the database 243, and to transmit timing data indicating the onset of the second time window to the timing support tool via a second communication link 260, wherein the timing support tool 110, 250, 500, 600 is arranged to support timing of the concrete processing operation in the second area B based on the timing data.

FIG. 7 is a flow chart illustrating a method for processing concrete which summarizes the above discussion. The method comprises determining S1 a temperature and/or a moisture level of a maturing concrete slab in a first area A by one or more concrete sensors 220, and transmitting sensor data associated with the temperature and/or moisture level via respective first communication links 230 to a data processing system 240, determining S2 a concrete maturity level for the first area A based on the sensor data, and determining an onset of a first time window 301 for processing concrete in the first area A, based on the concrete maturity level and on data associated with the concrete processing operation obtained from a database 243, and providing S3 the determined onset of the first time window to a timing support tool 110, 250, 500, 600 via a second communication link 260 for supporting timing of the concrete processing operation in the first area A.

FIG. 8 schematically illustrates, in terms of a number of functional units, the general components of a control unit 110. Processing circuitry 810 is provided using any combination of one or more of a suitable central processing unit CPU, multiprocessor, microcontroller, digital signal processor DSP, etc., capable of executing software instructions stored in a computer program product, e.g. in the form of a storage medium 830. The processing circuitry 810 may further be provided as at least one application specific integrated circuit ASIC, or field programmable gate array FPGA.

Particularly, the processing circuitry 810 is configured to cause the device 180 to perform a set of operations, or steps, such as the methods discussed in connection to FIG. 7 and the discussions above. For example, the storage medium 830 may store the set of operations, and the processing circuitry 810 may be configured to retrieve the set of operations from the storage medium 830 to cause the device to perform the set of operations. The set of operations may be provided as a set of executable instructions. Thus, the processing circuitry 810 is thereby arranged to execute methods as herein disclosed.

The storage medium 830 may also comprise persistent storage, which, for example, can be any single one or combination of magnetic memory, optical memory, solid state memory or even remotely mounted memory.

The device 110 may further comprise an interface 820 for communications with at least one external device. As such the interface 820 may comprise one or more transmitters and receivers, comprising analogue and digital components and a suitable number of ports for wireline or wireless communication.

The processing circuitry 810 controls the general operation of the control unit 110, e.g., by sending data and control signals to the interface 820 and the storage medium 830, by receiving data and reports from the interface 820, and by retrieving data and instructions from the storage medium 830.

FIG. 9 illustrates a computer readable medium 910 carrying a computer program comprising program code means 920 for performing the methods illustrated in FIG. 7, when said program product is run on a computer. The computer readable medium and the code means may together form a computer program product 900.

1. A concrete processing system for determining an onset of a first time window for a concrete processing operation in a first area, the system comprising one or more concrete sensors, a data processing system and a timing support tool,
wherein the one or more concrete sensors are configured to determine a temperature and/or a moisture level of a maturing concrete slab in the first area, and to transmit sensor data indicating the temperature and/or moisture level via respective first communication links to the data processing system,
the data processing system comprising a database and processing circuitry configured to determine a concrete maturity level for the first area based on the sensor data, to estimate the onset of the first time window based on the concrete maturity level and on data associated with the concrete processing operation stored in the database and to transmit timing data indicating the onset of the first time window to the timing support tool via a second communication link,
wherein the timing support tool is arranged to support timing of the concrete processing operation in the first area based on the timing data.

2. The concrete processing system according to claim 1, wherein the data associated with the concrete processing operation comprises data associated with a particular type of concrete processing tool or machine and/or a particular type of concrete processing operation.

3. The concrete processing system according to claim 1, wherein the timing support tool comprises display means arranged to indicate onset of the first time window to a user of a concrete processing tool in the first area.

4. The concrete processing system according to claim 1, wherein the processing circuitry is arranged to trigger the concrete processing operation in the first area at the onset of the first time window by the timing support tool.

5. The concrete processing system according to claim 1, wherein the data processing system is also arranged to determine a cessation of the first time window based on the concrete maturity level and on data associated with the concrete processing operation stored in the database, and to transmit timing data indicating the cessation of the first time window to the timing support tool via the second communication link.

6. The concrete processing system according to claim 1, wherein the processing circuitry is arranged to generate a warning signal, and/or to prevent concrete processing operation in the first area by the timing support tool before the onset of the first time window and/or after cessation of the first time window.

7. The concrete processing system according to claim 1, wherein the data processing system is configured to determine the concrete maturity level by indexing a maturity level table stored in the database using the sensor data.

8. The concrete processing system according to claim 1, wherein the data processing system is configured to determine the onset of the first time window in dependence of a pre-determined maturity level requirement of the concrete processing operation, or is configured to obtain a recipe of the concrete used in the concrete slab in the first area, and to determine the concrete maturity level in dependence of the recipe.

9. (canceled)

10. The concrete processing system according to claim 1, wherein the data processing system is configured to obtain weather forecast data and/or data from one or more weather stations deployed in connection to the first area, and to predict a future concrete maturity level in dependence of the weather forecast data and/or in dependence of the data from the one or more weather stations, or
wherein the data processing system is configured to generate extrapolated temperature and/or moisture level data from the sensor data, and to predict a future onset and/or a future cessation of the first time window.

11. (canceled)

12. The concrete processing system according to claim 1, wherein the database is configured to be populated by data from empirical testing of concrete processing operations.

13. The concrete processing system according to claim 1, configured for also determining an onset and/or a cessation of a second time window for a concrete processing operation in a second area,
wherein the one or more concrete sensors are configured to determine a temperature and/or a moisture level of a maturing concrete slab in the second area, and to transmit second sensor data indicating the temperature and/or moisture level via respective first communication links to the data processing system,
wherein the data processing system is configured to determine a concrete maturity level for the second area based on the second sensor data, to estimate the onset of the second time window based on the concrete maturity level and on data associated with the concrete processing operation stored in the database, and to transmit second timing data indicating the onset of the second time window to the timing support tool via the second communication link,
wherein the timing support tool is arranged to support timing of the concrete processing operation in the second area based on the second timing data.

14. The concrete processing system according to claim 1, configured to determine at least respective onsets of time windows for any of panning, floating, and finishing power troweling concrete processing operations, and an early entry sawing concrete processing operation.

15. The concrete processing system according to claim 1, comprising a plurality of concrete sensors arranged distanced from each other over the first area, wherein the processing circuitry is configured to determine the concrete maturity level for the first area based on the concrete sensor reporting data indicating the lowest maturity level, or to estimate the onset of the first time window based on a variation in the sensor data from the plurality of distanced concrete sensors.

16. (canceled)

17. The concrete processing system according to claim 1, wherein the one or more concrete sensors are deployed at a depth between 2-15 cm from the concrete surface.

18. The concrete processing system according to claim 1, wherein the data processing system is arranged to generate log data pertaining to the first time window and to the concrete processing operation,
to generate a warning and/or an alarm signal in case the concrete processing operation is performed outside of the first time window, or
to generate a receipt and/or a certificate in case the concrete processing operation is performed within the first time window.

19. (canceled)

20. (canceled)

21. The concrete processing system according to claim 1, wherein the data associated with the concrete processing operation stored in the database comprises information related to previous concrete processing operations,
wherein the previous concrete processing operations are processing operations using a particular type of concrete processing tool or machine and/or a particular type of concrete processing operation, or
wherein the information related to the previous concrete processing operations comprises one or more statistics of onset of the processing operation with corresponding concrete temperature and/or a moisture level.

22. (canceled)

23. (canceled)

24. A data processing system for determining an onset and/or a cessation of a first time window for a concrete processing operation in a first area,
wherein the data processing system is configured to receive sensor data associated with a temperature and/or moisture level of a maturing concrete slab in the first area,
the data processing system comprising a database and processing circuitry configured to determine a concrete maturity level for the first area based on the sensor data, and to determine an onset and/or a cessation of a first time window based on the concrete maturity level and on data associated with the concrete processing operation stored in the database.

25. A timing support tool arranged to connect to a data processing system, and to obtain timing data associated with an onset and/or a cessation of a first time window for a concrete processing operation in a first area from the data processing system via a second communication link.

26. The timing support tool according to claim 25, arranged to obtain timing data associated with an onset and/or a cessation of a second time window for a concrete processing operation in a second area from the data processing system via the second communication link.

27. (canceled)

28. A method for processing concrete, the method comprising
determining a temperature and/or a moisture level of a maturing concrete slab in a first area by one or more concrete sensors, and transmitting sensor data associated with the temperature and/or moisture level via respective first communication links to a data processing system,
determining a concrete maturity level for the first area based on the sensor data, and determining an onset of a first time window for processing concrete in the first area, based on the concrete maturity level and on data associated with the concrete processing operation obtained from a database,
providing the determined onset of the first time window to a timing support tool via a second communication link for supporting timing of the concrete processing operation in the first area.

29. The method of claim 28, further comprising deploying the one or more concrete sensors at a depth configured in dependence of a particular concrete processing operation, and wherein the depth is between 2-15 cm.

30. (canceled)
